(19) United States
(12) Patent Application Publication
(10) Pub. No.: US 2007/0270752 A1
(43) Pub. Date: Nov. 22, 2007

(54) Multifunctional instrument introducer

(76) Inventor: Denis LaBombard, Georgetown, MA (US)

Correspondence Address: Denis LaBombard;Sandbox LLC, PO Box 18, Georgetown, MA, 01833, US

(21) Appl. No.: 11/804,401

(22) Filed: May 18, 2007

Related U.S. Application Data
(60) Provisional application No. 60/801,301, filed on May 18, 2006.

(51) Int. Cl. A61M 5/178

(52) U.S. Cl. 604/164.01

(57) ABSTRACT

A flexible instrument introducer has a central channel and several peripheral channels in its outer wall for passage of instruments and other devices. It is assembled from three or more cylindrical elements, snapped together to give a limited degree of bending at each joint. The bending is confined to one or a few planes. The tube formed from the cylinders is stabilized and sealed by a flexible tube surrounding it, and said flexible tube confines controls and devices placed in the peripheral channels. The flexible tube also permits the maintenance of sterility and/or application of vacuum in the central and peripheral channels. The introducer may have an adaptor which keeps the distance from the proximal end to a device constant while the overall introducer bends.

### PRIORITY

This application claims the benefit of the priority of U.S. provisional application 60/801,301, filed May 18, 2006, which is hereby incorporated in its entirety by reference where permitted.

### FIELD OF THE INVENTION

The present invention relates to a multifunction device for introducing endoscopic devices and other surgical instruments into the body cavity, and, more particularly, to the design of a multi-lumen highly torque-able yet flexible device which can guide an endoscope or surgical instrument to target tissue within the body. This device includes multiple working channels formed in the wall of the device, and a clear, unobstructed, central axial channel which is capable of providing passage of one or more instruments to an internal site. The working channels can also be used to secure the device to a tissue wall. A sterile field may be provided within the central channel. Other devices, such as a tissue closure device, and be pre-positioned on the device. The device is distinctive in being both flexible, able to bend in at least one plane, and also “torqueable”, i.e. able to be precisely and reproducibly rotated.

REFERENCES CITED

| | | |
|---|---|---|
| 20060058582 | March, 2006 | Maahs; Tracy D.; et al. |
| 20060025654 | February, 2006 | Suzuki; Keita; et al. |
| 20050107664 | May, 2005 | Kalloo, Anthony Nicolas; et al. |
| 6974411 | December, 2005 | Belson |
| 6960163 | November, 2005 | Ewers, et al. |
| 6761685 | July, 2004 | Adams, et al. |
| 6179776 | January, 2001 | Adams, et al. |
| 4651718 | March 1987 | Collins, et al. |
| 4290421 | September, 1981 | Siegmund |
| 3266059 | August, 1966 | Stelle |

### OTHER REFERENCES

Modlin I M. Perspectives and reflections on integrated digestive surgery. Best Practice & Res Clin Gastroenterol 2002; 16(6):885-914.
Cotton P B. Interventional gastroenterology (endoscopy) at the crossroads: a plea for restructuring in digestive diseases. Gastroenterology 1994; 107:294-99.
Vitale G C, Davis B R, Tran T C. The advancing art and science of endoscopy. Amer J Surg 2005; 190(2):228-33.
Jagannath S B, Katsevoy S V, Vaughn C A, Chung S S, Cotton P B, et al. Peroral transgastric endoscopic ligation of fallopian tubes with long-term survival in a procine model. Gastrointest Endosc 2005; 61:449-53.
Fritscher-Ravens C A, Mosse C A, Mukherjee D, et al. Transgastric gastropexy and hiatal hernia repair for GERD under EUS control a porcine model. Gastrointest Endosc 2004; 509:1106.
Ponsky J L. Gastroenterologists as surgeons: what they need to know. Gastrointest Endosc 2005; 61(3):454.
SGE/SAGES Working group on Natural Orifice Translumenal Endoscopic Surgery. Gastrointest Endosc 2006: 63; 199-203

### BACKGROUND OF THE INVENTION

An endoscope is a flexible medical device for insertion into a body passageway or cavity that enables an operator, positioned at a remote external location, to view a site internal to the patient's body. It is often desirable to perform certain surgical procedures at internal sites, and to be able to view the site during the procedure. For historical reasons, in general, an endoscope now may comprise a long flexible tubular member equipped with, for example, a miniature viewing device, an illumination device, and working channels. The endoscope has a proximal end that remains external to the patient and a distal end having an endoscope tip for insertion into a body cavity of the patient. In this discussion, we will often refer to a device containing both visualization means, and additional spaces for passage of instruments, as an endoscope. (We also call the device an “instrument introducer”, especially when it does not necessarily contain visualization means.)

In a typical endoscope, an illumination device of the endoscope includes a lens at an endoscope tip. The lens is positioned against the illumination device proximate to a viewing device. Light emanates from the lens to enable the viewing device to capture images in the body cavity, and electrically or optically transmit the images through the endoscope for display at an external monitor.

Once viewing the images, the endoscope operator may insert one or more surgical instruments through working channels within the overall diameter of the endoscope to perform an endoscopic procedure at the internal body cavity site. These endoscopic procedures may include, for example, snaring ligation, counter ligation, suturing, cutting, stenting, injections, or biopsies of particular internal areas of the patient's body. This instrument in numerous configurations and designs has become the workhorse of surgical procedures in the field of gastroenterology.

Natural Orifice Translumenal Endoscopic Surgery (NOTES) is a new and developing extension of current surgical methods in the field of flexible endoscopy. In a NOTES procedure, an endoscope is used to pass through a natural orifice into a natural luminal space, for example the stomach. The endoscope is then located to a desired location on the wall of the natural lumen, where it is used to create a port through the wall. Next, the endoscope can view the translumenal space and perform one or more procedures there. Then the port is closed, and the device is removed. The system is advantageous for certain types of surgery where normal trans-dermal operative procedures require extensive repositioning of organs to reach the target site. Lumenal walls often heal very quickly, and cutting of muscles can be avoided.

The NOTES technique to date, as evidenced by a number of recent publications, has allowed appendectomy, tubal ligation, gastroenterostomy, and even cholecystectomy. Surgeons, in the absence of specific devices designed to easily generate location and securing capability within the gastric system, have in some cases used an existing simple gastric tube as an endoscope instrument guide for performing a NOTES procedure. Such delivery devices are not especially efficacious for locating an access site, securing the site, maintaining a sterile field during the procedure, and effectively closing the incision upon leaving the surgical site. Improved devices are required to advance NOTES and similar techniques.

An example of a first-generation device is the Gardus™, manufactured by U.S. Endoscopy Corp. Gardus™ is not a torqueable device except when deployed. (Herein, a “torqueable” device is one than can be rotated about its long axis without creating a rotational displacement along its length. Dry spaghetti is torqueable; wet spaghetti, like the Gardus and the USGI instruments (when not axially compressed), is not.) It is a flexible device that relies on the endoscope placed within to provide directional control. It is then stiffened once it has been put into position. It does not have an integral means for closing tissue incisions.

A more appropriate methodology which supports the surgeon's needs in management of the sterile field is to provide a multifunctional type on instrument which could allow multiple instruments to be present within the gastric system to facilitate the NOTES procedures.

U.S. Pat. No. 6,761,685, Adams et al., describes a sheath based system for the delivery of multiple instruments, including an endoscope, placed inside a central channel to guide them. The device is said to facilitate the delivery of multiple instruments to the endoscope tip for tissue manipulation. However, it is limited in other desired capabilities. The construct is a flexible sheath based system in design, where the “sheath” is a thin membrane-like construct. It cannot structurally stand alone, nor support the use of vacuum. Since it is a sheath, it has no structural strength along its axial length other than what the endoscope instrument provides. This is a disadvantage in that a structural channel cannot be maintained without the endoscope, therefore the exchange of instruments within the sheath is not possible, nor is passing the endoscope beyond the tip element of the sheath based system also unattainable.

It is an object of this invention to demonstrate an improved structural geometry which will overcome these deficiencies while still maintaining flexibility, which can be manipulated by an endoscope within the central channel. Further, the rigidity of a preferred embodiment and its enhanced torque and compression resistance properties will allow the endoscope to pass beyond the distal tip of the device and facilitate the use of vacuum, a significant improvement over the current art.

U.S. Pat. No. 4,651,718, Collins et al., describes a construct which includes a structural mechanism scheme for maintaining a stiff central core; however this construct includes elements which by design occupy the central portion of the device where an endoscope instrument would need to pass. A multifunction al delivery system based on such an approach would severely limit the size of the instruments that could be passed within the central portion, because of the requirements for securing the interacting pivot like elements.

It is an object of this invention to define a much improved superior embodiment to U.S. Pat. No. 4,651,718 Collins, where the hinge like elements interlock, and the central portion of the device is at a maximum size, providing a large clear annular central unobstructed volume for instruments to pass.

U.S. Pat. No. 6,960,163 (Ewers et al), U.S. Pat. No. 6,974,411 (Belson) and U.S. Patent Application 2006-0058582 (Maahs et al.) all describe art which includes various interlocking elements with generally spherical or pseudo-spherical slideable surfaces which can be made flexible and then made rigid using a plurality of tensioning members arrayed about the axial wall. These tensioners provide a locking force to the interfacing surfaces when the tension members are activated.

These embodiments provide a clear central annular channel, and when locked may also provide a highly torque-able assembly. However, the interlocking members used in these embodiments must have the capability of sliding surfaces rotationally by each other in a pivot like scheme to flex into position. The pivot axis is located transverse to the longitudinal central axis, and the pivot point is the intersection of the defined axes. The pivoting requirement and placement is a serious constraint and impediment to the addition of multiple annular channels within the outer wall for use in the delivery of instruments such as is the object of the preferred embodiment of the present invention.

The use of tension wire-like members within the outer wall of these prior art embodiments to generate the rigidity of structure compresses the interlocking elements to prevent movement. The interlocking elements, having a generally spheroidal interface, are intend to nest together to generate flexure resistance by the friction of the engaging surfaces, which also has the net effect of distorting the empirical spherical shape and displacing the lumen in the walls. Such constructs are deficient in performance in passing instruments through the wall when locked, for if instruments were to be delivered within the structural walls of these embodiments, the peripheral axial path defined by the walls can become pinched and closed as the element interface surfaces are flexed and moved slideably by each other. Effectively, instruments can pass only in the center channel.

Furthermore, efforts to mitigate such pinching effect by removing material to clear away the pinching and high friction interference portion of the slideable surfaces then reduces the annular structural robustness, resistance to slippage and collapse resistance of the assembly, which must by design sustain a significant compressive force placed on each element by the locking members to attain the rigidity for transmitting torque in the locked state.

Thus, the prior art does not provide a highly torqueable introducer endoscopic instrument which does not rely on friction between slideable surfaces to provide torqueability. Moreover, the prior art does not provide a torqueable introducer endoscopic instrument which provides useable passages in its walls for passage of control wires and other devices through its walls, in addition to passage through a central lumen. The prior art also does not describe a highly torqueable introducer endoscopic instrument in which sterility can be maintained in the central lumen during deployment to, and use at, a site internal to the body.

### SUMMARY OF THE INVENTION

It is an object of this invention to demonstrate an improved embodiment of interlocking elements which will provide a highly torqueable device without the use of friction interacting surfaces. It is also an object of the present invention to demonstrate an improved embodiment for delivering and endoscopic instrument which includes additional instrument pathways located within the structural wall, which are not affected by flexure between elements, thus overcoming limitations of the previous art and providing additional instruments to the target site which do not occupy the central volume of the device. Such instruments may be available at any selected position along the instrument and does not require utilizing the central axial volume, nor partitioning it.

It is an object of this invention to provide a multifunctional instrument introducer which will allow the passage of endoscopic instruments through a large unobstructed central axial channel (“central channel”) into a body cavity, to and beyond a surgical access site located within said body cavity.

It is an object of this invention to provide an introducer which will have means for maintaining position in relation to said surgical access site, while a procedure is conducted.

Preferably, the introducer will allow a secure uninterrupted contact interface and manipulative control of said surgical access site tissues.

It is an object of this invention to provide a means of independently closing and securing said surgical access site tissues upon removal of surgical instruments residing within said instrument.

It is an object of this invention to define an integrated multifunctional device embodiment which includes manipulation and control of multiple instruments residing within a single embodiment that meets the requirements of an instrument delivery device to execute a NOTES procedure, wherein an identified membrane or tissue within the body is safely located, secured, managed, penetrated to generate access through, maintained and then subsequently securely closed, while maintaining a sterile field for the purpose of introducing surgical instruments, devices or medicaments, singly or in multiple, to and beyond said membrane or tissue location within said body.

Such clinical procedures may be part of a surgical protocol to conduct surgical activities or therapeutic procedures utilizing surgical instruments and commercially available endoscopes singly or together in concert with and through said multifunctional instrument introducer device.

It is an object of this invention to provide a flexible introducer, optionally with endoscopic features, which can be provided in a sterile condition and maintain sterility in its internal channels and central lumen during navigation to a site internal of the body and execution of a procedure.

It is an object of this invention to define a unique cost effective method of constructing a flexible tube multifunctional instrument introducer, the capabilities as described above which can be tailored in size and construct to meet the requirements of numerous types of surgical, therapeutic and/or diagnostic procedures.

It is also an object of this invention to define a unique low-cost method of constructing a flexible tube multifunctional instrument introducer with the ability to be introduced into the body cavity atraumatically, further comprising one or more of an endoscope and an on-board optical guidance system.

### BRIEF DESCRIPTION OF THE FIGURES

FIG. 1 shows an isometric view of the multifunctional instrument introducer in an embodiment of the present invention.

FIG. 2 is a planar view of the multifunctional instrument introducer in an embodiment with the outer sheath hidden to reveal the interlocking multi-lumen tubular elements residing within.

FIG. 3 illustrates the multifunctional instrument introducer in a shortened assembly with a functionally minimum number of the interlocking multi-lumen tubular elements and the outer sheath partially cut-away to reveal said elements.

FIG. 4A illustrates an interlocking multi-lumen tubular element in axial view. FIG. 4B illustrates an interlocking multi-lumen tubular element in a side view showing the detail of interlocking features. FIG. 4C is a cross sectional view of FIG. 4A. FIG. 4D is an isometric view of the interlocking element.

FIG. 5A illustrates the interlocking multi-lumen tubular elements partially cut away for clarity to reveal feature relations and feature orientation. FIG. 5B is an enlarged detail of the locking feature shown in FIG. 5A. FIG. 5C is an axial view of FIG. 5A with outer sheath shown to illustrate the interlocking multi-lumen tubular element geometry relationship. FIG. 5D is an enlarged detail of a single peripheral instrument channel shown in FIG. 5C

FIGS. 6A, 6B, 6C and 6D illustrate additional features and relationships shown in FIG. 3 in an exploded view and uses additional cut away portions for a number of elements to assist the description of embodiment function.

FIG. 7A FIG. 7B and FIG. 7C illustrate a peripheral instrument channel suture “t” stay needle assembly instrument, the related components and the function in detail.

FIG. 8A, FIG. 8B, FIG. 8C FIG. 9 and FIG. 10 illustrate the general operational sequence that would be employed to locate anchor and access a surgical site. FIG. 8A illustrates the multifunctional instrument introducer which has been located at the surgical site with the suture “t” stay needle assembly advanced and engaging tissue, representing the start of a NOTES surgical procedure. FIG. 8B is an enlarged illustration view of the multifunctional instrument introducer distal end showing the suture “t” stay needle assembly distal end detail. FIG. 8C is an enlarged illustration view of the multifunctional instrument introducer distal end showing the suture “t” stay now deployed and the distal instrument end anchored and secured to the tissue.

FIG. 9 is an illustration of the multifunctional instrument introducer at the end of a NOTES procedure where the endoscope is residing within the instrument and the incision now needs to be closed, showing the suture “t” stays in the deployed condition and the operation sequence to deploy a the self closing tissue fastener to close surgical site.

FIG. 10 is an illustration of the self closing tissue fastener in the deployed position with the multifunctional instrument introducer being retracted which represents the condition and location of the multifunctional instrument introducer and self closing tissue fastener at the end of a ‘NOTES’ procedure just as the multifunctional instrument introducer is to be removed.

### DESCRIPTION OF THE INVENTION

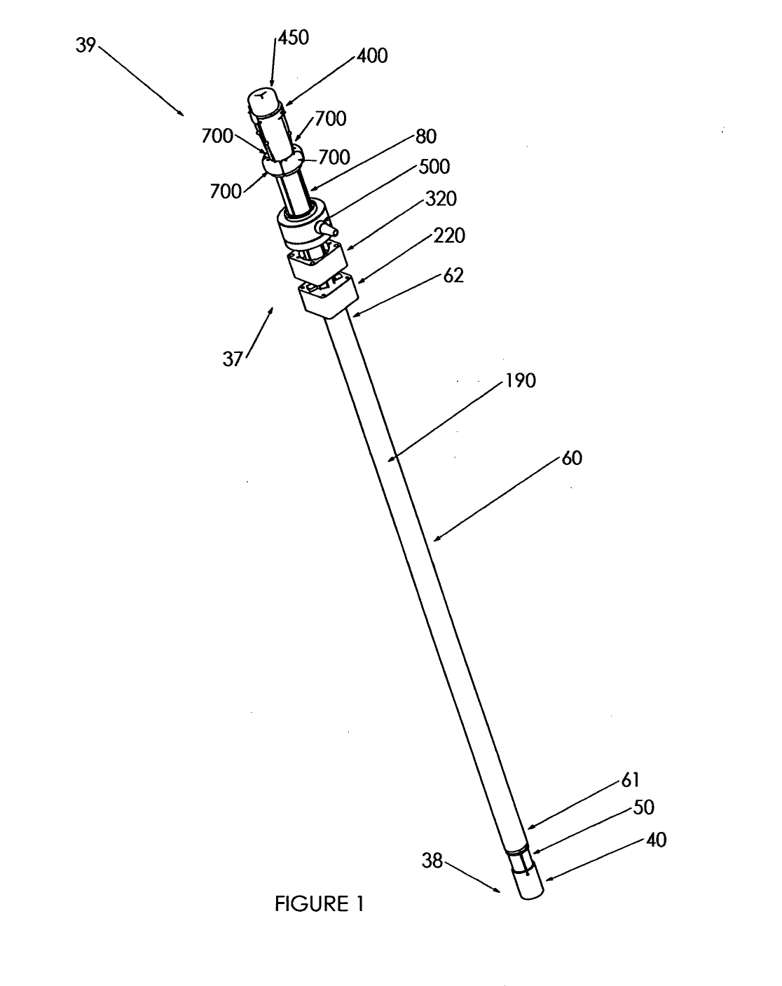

FIG. 1 shows an isometric view of the multifunctional instrument introducer in the preferred embodiment of the present invention. Referring to FIG. 1, multifunctional instrument introducer 39 is comprised of a multifunctional instrument introducer distal end detail 38, a multifunctional instrument introducer control end 37, and an endoscope delivery tube assembly 60 shown in this view as covered by outer sheath 190, and comprised of an endoscope delivery tube assembly distal end 61 and an endoscope delivery tube assembly proximal end 62. On the multifunctional instrument introducer 39, shell element 40 slides in a sealable manner on tubular connecting element 50 which is sealably engaged with endoscope delivery tube assembly distal end 61. Residing within shell 40 and not shown here is a self closing tissue fastener, intended for delivery by the introducer 39. Optionally, more than one tissue fastener could be carried in this manner.

Endoscope delivery tube assembly proximal end 62 is sealably engaged with distal collar assembly 220 residing and sealably connected to control end tubular member 80 which runs the full length of multifunctional instrument introducer control end 37.

Residing on control end tubular member 80 are the following additional assemblies and elements, each which have a hollow central core to allow control end tubular member 80 to pass through the feature, and/or for the feature to slide upon tube 80 without impediment:

distal collar assembly 220; self closing tissue fastener firing collar assembly 320; rotary vacuum assembly 500; a radial array shown in the preferred embodiment of four suture “t” stay needle assemblies 700; proximal suture collar assembly 400; and lastly endoscope seal 450, residing at the extreme proximal end of multifunctional instrument introducer 39. The functions of these assemblies will be discussed in more detail below.

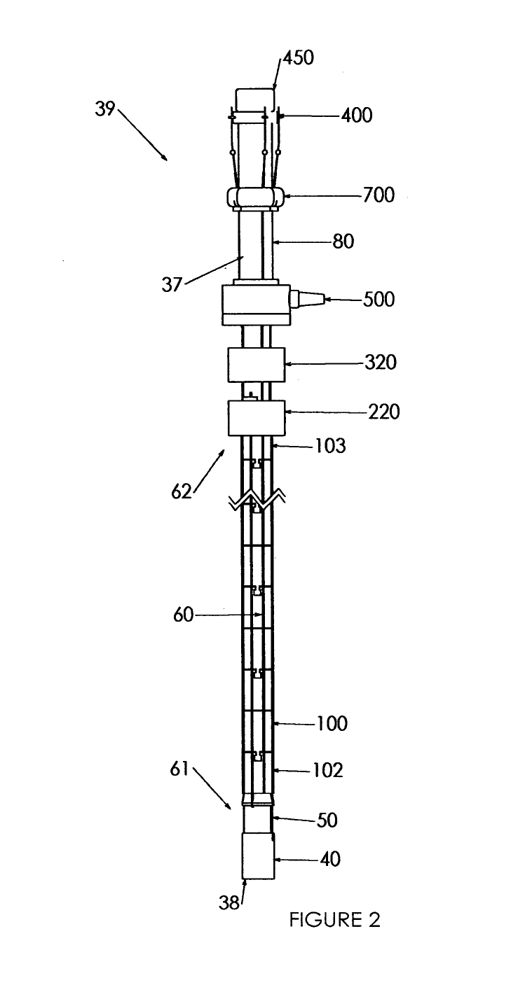

FIG. 2 shows a planar view of the multifunctional instrument introducer 39 in the preferred embodiment with the outer sheath 190 (previously shown in FIG. 1) hidden to reveal the multiple count of interlocking multi-lumen tubular elements 100. Multifunctional instrument introducer 39 is shown comprised of a multifunctional instrument introducer distal end detail 38, a multifunctional instrument introducer control end 37. Endoscope delivery tube assembly 60 shown in detail in this view is comprised of multiple interlocking multi-lumen tubular elements 100, and an interlocking multi-lumen tubular distal transition element 102 at the endoscope delivery tube assembly distal end 61 and an interlocking multi-lumen tubular proximal transition element 103 at the endoscope delivery tube assembly proximal end 62 respectively.

Shell element 40 (bottom) is sealably sliding on tubular connecting element 50 which is sealably engaged with interlocking multi-lumen tubular distal transition element 102 at endoscope delivery tube assembly distal end 61. Interlocking multi-lumen tubular proximal transition element 103 at endoscope delivery tube assembly proximal end 62 is sealably engaged by distal collar assembly 220 residing and sealably connected to control end tubular member 80 which engages multi-lumen tubular proximal transition element 103 at its distal end and runs the full length of multifunctional instrument introducer control end 37.

Residing on control end tubular member 80 is the following additional assemblies and elements are shown: self closing tissue fastener firing collar assembly 320, Rotary vacuum assembly 500, a radial array of four suture “t” stay needle assemblies 700, followed by Proximal suture collar assembly 400 and Endoscope seal 450 residing on control end tubular member 80 at the instrument proximal end.

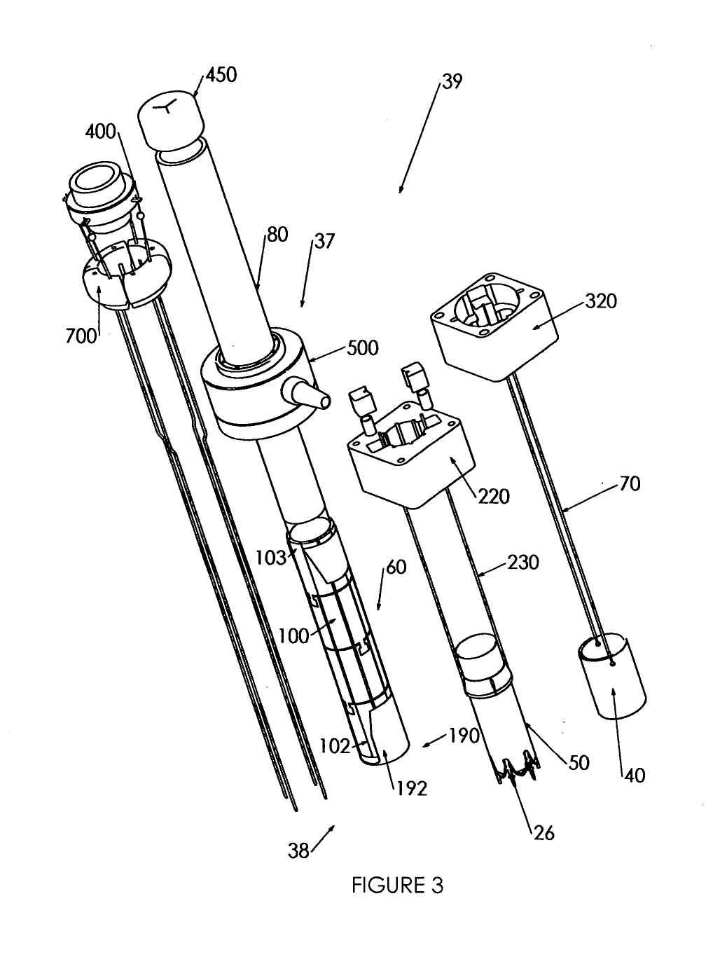

FIG. 3, an exploded view, illustrates in an isometric view the multifunctional instrument introducer 39 in a shortened assembly with a functionally minimum count of the interlocking multi-lumen tubular elements 100 and the outer sheath 190 partially cut-away to reveal more detail. Multifunctional instrument introducer 39 is shown in a shorter length embodiment, maintaining all functional aspects and relations of the preferred embodiments shown in FIGS. 1 and 2 is comprised of a multifunctional instrument introducer distal end detail 38, a multifunctional instrument introducer control end 37.

Endoscope delivery tube assembly 60, shown in this view for the purpose of defining a typical minimum length embodiment with full functionality, is comprised of just two interlocking multi-lumen tubular elements 100, an interlocking multi-lumen tubular distal transition element 102 at endoscope delivery tube assembly distal end 61, and an interlocking multi-lumen tubular proximal transition element 103 at endoscope delivery tube assembly proximal end 62 respectively.

It should be clear to one skilled in the art and from the illustrations in FIGS. 1, 2 and 3 that the length of the instrument can be totally variable and tailored for specific surgical applications by specifying the count of the interlocking multi-lumen tubular element 100 for developing a given working length.

Following the description sequence used in FIGS. 1 and 2, shell element 40 is connected by pull wires 70 to self closing tissue fastener firing collar assembly 320, which is sealably sliding on tubular connecting element 50, and connected by length control wires 230 to distal collar assembly 220 which has a self closing tissue fastener 26 located at its distal end. Tubular connecting element 50 is sealably engaged with interlocking multi-lumen tubular distal transition element 102 at endoscope delivery tube assembly distal end 61 Outer sheath 190 shown in a cutaway view encapsulates interlocking multi-lumen tubular element 100, interlocking multi-lumen tubular proximal transition element 103 and interlocking multi-lumen tubular distal transition element 102, where outer sheath outer surface 192 provides a smooth seamless a-traumatic outer surface interface to body tissue during use.

Interlocking multi-lumen tubular proximal transition element 103 at endoscope delivery tube assembly proximal end 62 is sealably engaged by distal collar assembly 220 residing and sealably connected to control end tubular member 80 which engages multi-lumen tubular proximal transition element 103 at its distal end and runs the full length of multifunctional instrument introducer control end 37. Residing on control end tubular member 80 is the Rotary vacuum assembly 500 and endoscope seal 450. The radial array of four suture “t” stay needle assemblies 700, and the proximal suture collar assembly 400 also residing on control end tubular member 80 is illustrated in an exploded view configuration.

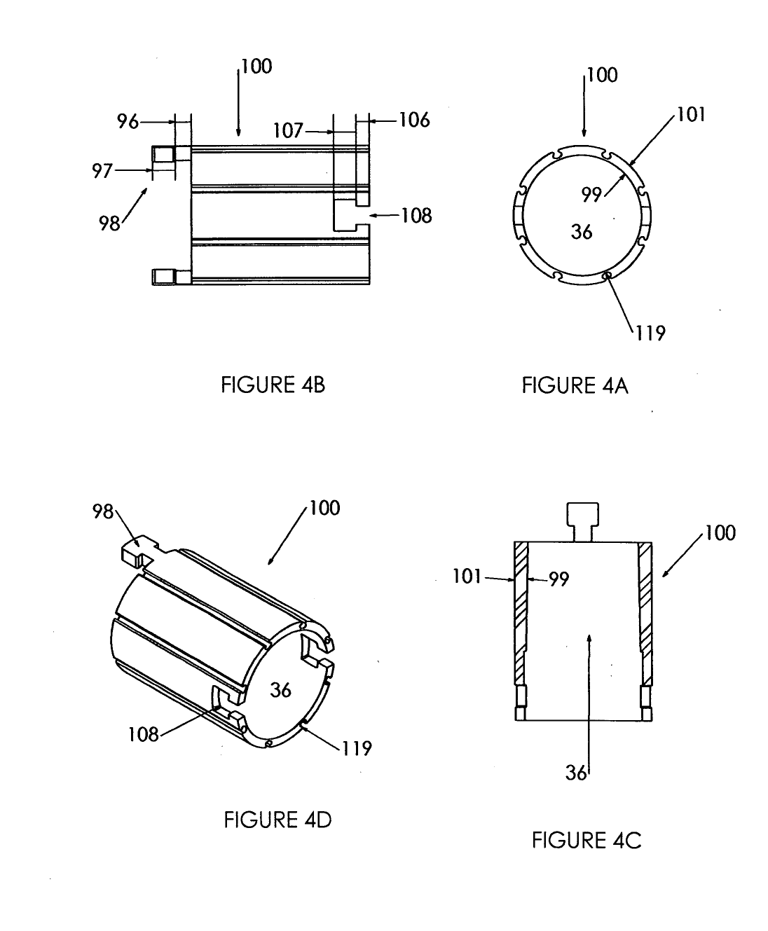

FIGS. 4A-4D will now illustrate the detail of interlocking multi-lumen tubular element 100. The detailed properties of element 100 are important in producing the improved functional properties of the instrument. Interlocking multi-lumen tubular element 100 is comprised of an inner surface 99 and an outer surface 101, which define a relatively thin shell surrounding the clear unobstructed central volume 36 along the axial length of the instrument. Within the walls of the tubular member 100 are one or more axial peripheral instrument channels 119. As described in more detail below, the instrument channels 119 can carry any of a variety of steering wires, fastener control wires, affixation devices, fiber optics, and the like.

At one end of interlocking multi-lumen tubular element 100 is shown the male interlocking geometry 98 which is defined in the preferred embodiment as consisting of a male interlocking geometry neck having a length 96, and a male interlocking geometry head having a length 97. At the other end of the element is female interlocking geometry 108 which is defined in the preferred embodiment as consisting of a female interlocking geometry neck having length 106, and a female interlocking geometry head having length 107. Such interlocking features as described are intended to securely engage and hold a number of interlocking multi-lumen tubular elements 100 to generate multifunctional instrument introducer 39 with defined performance properties.

In one embodiment of the present invention, interlocking multi-lumen tubular element 100 shown in FIGS. 4A-4D, the male interlocking geometry 98 features and female interlocking geometry 108 features are axially symmetric and male interlocking geometry 98 is located 90 degrees in axial rotation from female interlocking geometry 108 on each interlocking multi-lumen tubular element 100.

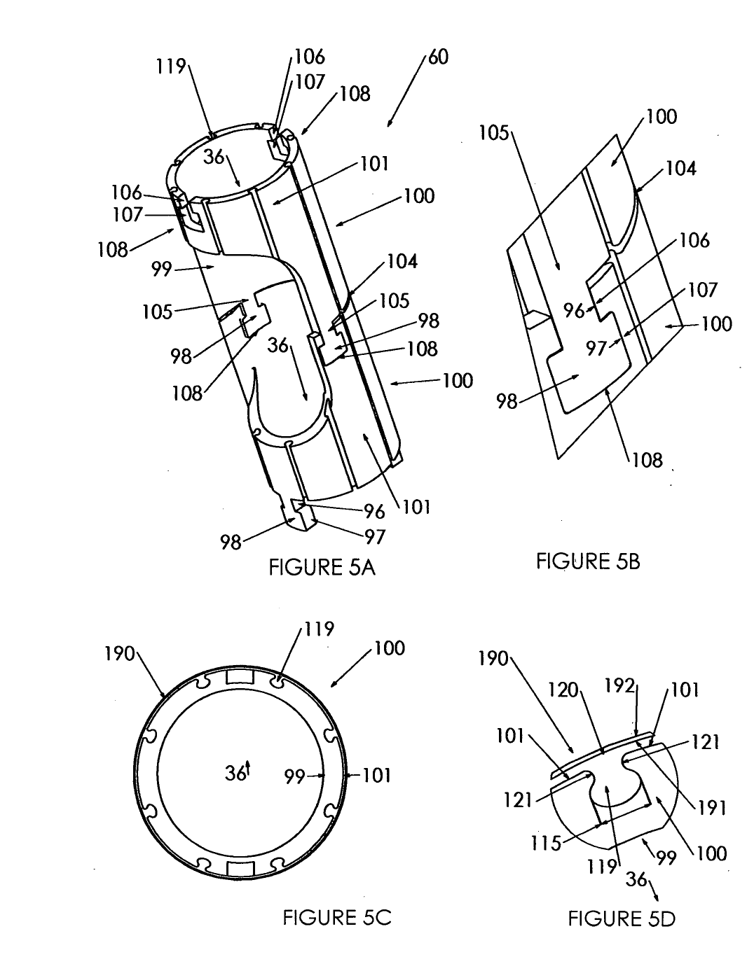

FIGS. 5A-5D, in conjunction with FIGS. 4A-4D previously described, illustrate detail and functional aspects of the endoscope delivery tube assembly 60 using multiple interlocking multi-lumen tubular elements 100. FIG. 5A is an isometric representative illustration of an engaged pair of the interlocking multi-lumen tubular elements (100) with the outer sheath (190) removed and is partially cut away in the central portion for clarity to reveal geometry relations and feature orientation. FIG. 5B is an enlarged detail of the interlocking features shown in FIG. 5A. FIG. 5C is an axial cross section view of FIG. 5A and includes the outer sheath (190) which is an integral part of the peripheral instrument channel (119). FIG. 5D is an axial enlarged detail view of FIG. 5C detailing a single peripheral instrument channel 119 and all related geometrical features required to create that channel.

In FIG. 5A, endoscope delivery tube assembly 60 is comprised of multiple interlocking multi-lumen tubular elements 100. The number of elements used in a device assembly creates the appropriate device length and bending capability of the multifunctional instrument introducer. For the purpose of describing the numerous features and relations of the interlocking multi-lumen tubular elements 100, two elements (100) are shown with portions cut away to reveal internal structure. In FIG. 5A, and axial view FIG. 5C, interlocking multi-lumen tubular element 100 is defined as a tubular structure with a clear unobstructed central volume 36 along the central axis. Located within the structural walls of interlocking multi-lumen tubular element 100 are peripheral instrument channels 119 running unobstructed along the length of the assembly. In the preferred embodiment shown in FIGS. 5A and 5C there is a count of eight peripheral instrument channel features 119. The number of channels 119 is variable, but can be an even number in all versions of element 100, and can be an odd number in certain versions, for example those having a 0 degree offset between male and female members, but not the one in FIGS. 4 and 5, where there is a 90 degree offset between male and female connectors, and the number of lumens 119 must be even.

In FIGS. 5A, 5B, 5C and 5D, one can clearly appreciate that there can be as few as one peripheral instrument channel 119, or as many peripheral instrument channels 119 as can be mechanically sustained within the tubular structural wall, and that any single or multiple combinations of peripheral instrument channel 119 features or spacing or array scheme can be grouped in a multitude of possible combinations or permutations of positions depending upon the net geometric shape of interlocking multi-lumen tubular elements 100, size and location of the male interlocking geometry 98 and female interlocking geometry 108 as well as the surgical functional and positional location requirements for instruments or control features to be placed within each peripheral instrument channel 119. In FIG. 5B, it can be seen how the channel 119 crosses the boundary between two adjacent elements 100.

FIG. 5D is an axial enlarged detail view of FIG. 5C which shows the detail of a single peripheral instrument channel 119. Peripheral instrument channel 119 is comprised of a peripheral instrument channel central volume 120 which includes peripheral instrument channel edge relief 121 on interlocking multi-lumen tubular element 100 which blends smoothly with the interlocking multi-lumen tubular element outer surface 101 of the interlocking multi-lumen tubular element 100 and the outer sheath inner surface 191 of outer sheath 190. The channel 119 as illustrated here is not typically cylindrical in profile, but generally oval or elliptical, with the long axis 115 of the oval being perpendicular to the radial direction of the element 100. This provides space for lateral movement of control wires and other devices moving within the channels 119, so that when the device 60 is bent about an axis perpendicular to axis 115 of channel 119, the wires and devices in the channels will be less likely to bind and fail to move.

There are distinct advantages in the manufacture of interlocking multi-lumen tubular elements 100 and the assembly of the multifunctional instrument introducer (39) to have the peripheral instrument channels (119 not fully enclosed and trapped within the tubular wall as is customary in the art for manufacturing typical multi-lumen components

First, this geometry by design can be easily modified to ensure that devices in the channels (119) will be able to move regardless of bend angle and instrument size.

The lumen geometry need not be constant along the axial length. It may be advantageous to increase the long axis (115) of the channel at each mating end surface of the interlocking tubular element 100. Such a geometry construct is well known in the art of injection molding of plastics and metallic materials where it is highly desirable to have draft angle on these features described to facilitate ejection from the mold. Such an addition of draft angle tapering from large at each mating end to a smaller dimension in the center, well known in the art would be an enhancement to the preferred embodiment and reduce device sliding friction. Thus both the central lumen and the peripheral channels will preferably be larger in diameter at the ends of cylindrical element 100, and narrowest at approximately the middle of the element.

Second, the tooling used for generating features 119, 120 and 121 is much more robust and durable where the lumen generating feature is attached to the tooling surfaces creating the interlocking multi-lumen tubular element outer surface 101 along its full axial length, rather than being a lumen generating core with only distal and proximal support. This improves the accuracy of generating the lumens 119 and reduces the cost of the tools Third, in the assembly of the instrument, long instruments or controls to be placed within the peripheral instrument channels 119 can be easily “snapped” laterally into the peripheral instrument channels 119 of the assembled tube 60 from the outside, rather than threaded in. Then tube 60's elements 100 are covered by the outer sheath 190. The primary function of sheath 190 is to serve as a constraint means, which retains the controls and other features in the channels 119. The outer sheath 190 is optionally and preferably made of a shrink-wrap material, which can be put into tight approximation to the outer wall 101 of the tube 60 to retain the wires and the like in the channels. This is much easier to assemble than assembly using controls that are threaded or snaked through the axial length of an enclosed lumen design of similar length. Such features provide significant cost advantages in manufacturing and assembly. In addition to shrink wrap, other materials can be used to provide a constraint means preventing the escape of wires and other devices from the channels 119. Other constraint means include, without limitation, polymeric and metallic mesh, braid, coils and bands, optionally including an airtight layer; self-sticking materials such as an adhesive tape and tubing cast in place. Each of these may be used alone, together, or in conjunction with shrink wrap or other impervous polymeric materials.

Furthermore, with such a design approach, in communicating the peripheral instrument channel 119 with the interlocking multi-lumen tubular element outer surface 101 wall, the actual volume of the peripheral instrument channel 119 can be significantly larger and less constrictive than enclosed lumen designs thus allowing for larger diameter instruments to be utilized in proportion to the interlocking multi-lumen tubular element 100 wall thickness.

The addition of features such as peripheral instrument channel edge relief 121 and non circular or non standard geometric shapes to generate the peripheral instrument channel central volume (120) can also reduce the friction within the peripheral instrument channel (119), further enhancing the slideability and control of the instruments placed within said channel. Selection of lubricious materials for the outer sheath (190) and interlocking multi-lumen tubular element (100) or placing lubricious coatings on the outer sheath inner surface (191) and related surfaces which create the peripheral instrument channels 119 are all capabilities and enhancements that fall within the scope of the present invention.

In FIG. 5A, at the axial distal end of the interlocking multi-lumen tubular element 100 is a pair of male interlocking geometries 98, each which include a male interlocking geometry neck length 96 and a male interlocking geometry head length 97 respectively. At the axial proximal end of FIG. 5A, showing the assembly of interlocking multi-lumen tubular elements 100, are a pair of matching female interlocking geometries 108 which include a female interlocking geometry head length 107, and a female interlocking geometry neck length 106 respectively. In a preferred embodiment of the present invention, shown in FIG. 5A, male interlocking geometry 98 features and female interlocking geometry 108 features are axially symmetric and male interlocking geometry 98 is located 90 degrees in axial rotation from female interlocking geometry 108 on a single interlocking multi-lumen tubular element 100.

Continuing with FIG. 5A and FIG. 5B, at the cut away centrally in the figure, is shown in detail the locking capability of interlocking multi-lumen tubular elements 100, Female interlocking geometry 108 and male interlocking geometry 98 is now in the engaged assembled condition. In this condition, male interlocking geometry head length 97 and female interlocking geometry head length 107 are intended to seamlessly and securely engage such that the connection between the two features is a snug fit, greatly limiting the axial movement between each of the interlocking multi-lumen tubular elements 100, and somewhat, but not completely, limiting the rotational movement.

Such a defined fit for the preferred embodiment thus confers to the multifunctional instrument introducer embodiment the ability to be highly torqueable. A high torque (highly torqueable) instrument has by design a minimum amount of rotational lag distal to proximal when the instrument is held by the proximal end and rotated within a body cavity. Such an attribute is also highly desire able in surgical procedures providing a high degree of positional control to the surgeon to correctly locate the various instruments located within the peripheral instrument channels 119 within the surgical field.

In a preferred embodiment, the length of the male interlocking geometry neck length 96 is defined in relation to the length of the female interlocking geometry neck length 106 such that an interlocking multi-lumen tubular element pivot gap 104 is created. Locating the interlocking multi-lumen tubular elements 100 in an axial alignment proximal to distal, the interlocking multi-lumen tubular element pivot gap 104 would now be annular in nature.

Having now defined a distal to proximal axial alignment condition for FIG. 5A or 5B, an interlocking geometry pivot axis 105 (best seen on FIG. 5B) can be defined as a virtual line drawn from the intersection of the midpoint of the tubular face of male interlocking geometry 98 and the midpoint of interlocking multi-lumen tubular element pivot gap 104 of the assembled embodiment located on one side of interlocking multi-lumen tubular element 100 to a matching location defined symmetrically located on the other side of the clear unobstructed central volume 36, each position shown in FIGS. 5A and 5B as feature 105, located at the tip of the arrow. (Think of a “virtual” pivot running from the tip of arrow 105 across the tube to the equivalent point on the other male element 98 of the particular element 100.). Such an interlocking geometry pivot axis 105 as defined would pass through the central centerline axis of the interlocking multi-lumen tubular elements 100, and would describe for the purposes of this application the definition of a planar pivoting motion. This relationship of features, feature gap and rotation axis due to symmetry and design has a planar pivoting capability with interlocking geometry pivot axis 105 located centrally on features 98 and 108 as previously demonstrated.

As the planar pivot motion occurs at interlocking geometry defined by the pivot axis 105, it is clear that interlocking multi-lumen tubular element pivot gap 104 becomes smaller on one side and larger on the other respectively, until at some point the pivot action will cause tubular walls to come into contact and thus stop any further motion in the direction taken. Such limitations of the planar pivot motion after a given angular translation are a distinct advantage to maintaining and passing instrumentation and control features within the clear unobstructed central volume 36 and through the multiple peripheral instrument channels 119. Additionally, these flexure limits also provide exceptional columnar and torque strength to the assembly, which further aids the surgeon when manipulating the instrument in an axial and or a combined axial and rotational manner.

Numerous geometrical relationships of interlocking and axial pivoting type geometries known in the art are also described by U.S. Pat. No. 6,960,163 (Ewers et al), U.S. Pat. No. 6,974,411 (Belson), and U.S. Patent Application 20060058582 (Maahs et al.). Such connecting and interfacing geometrical entities are intended in the present invention to link the interlocking multi-lumen tubular elements 100 tightly to each other while still providing a capability of a limited axial pivot through a central portion of that linking interface. Any geometry arrangement which may allow a similar function may also be employed in stand alone or integrated form.

In a preferred embodiment of the present invention, as shown in exploded view FIG. 3 and FIG. 5A, these connecting features are rotationally indexed by 90 degrees as each interlocking multi-lumen tubular element 100 is assembled, in that a male interlocking geometry 98 and female interlocking geometry 108 located within the same interlocking multi-lumen tubular element 100 is located 90 degrees apart as viewed from the central tubular axis, thus providing at least two unique independent planar pivot motions of flexure to the instrument when a total of at least 3 interlocking multi-lumen tubular elements 100 components are assembled.

One skilled in the art can appreciate that in a device 60, interlocking joints as shown in FIG. 3 or 5 may be designed to have a singular planar pivot motion direction alone for some distance and then for a further distance may be comprised of some other, optionally more complex spatial arrangement or series of arrangements and spacing of the pivot axes and element length, achieved by using transition elements and/or elements having different proportions and dimensions, could provide a specific preferred directional and flexure action at a specific axial length location.

Furthermore, one skilled in the art can also appreciate that instruments comprised of multiple designs and/or axial lengths of interlocking multi-lumen tubular element 100 components can therefore be defined with regions of varying curvature and planar pivot motion flexure which may be singularly planar or multi-planar or any combination thereof. Other such combinations of interlocking multi-lumen tubular element 100 configurations or designs may include but not be limited to the following examples.
Using separate distinct link entities to be embedded within the structural wall to join multiple interlocking multi-lumen tubular element 100 components. Such an independent link-like type component design may be separate or integral to element 100. For example, all of the connectors formed into the elements 100 could be female, and dog-bone or bar bell shaped connectors could be pressed into pairs of female connectors to join them. Likewise, pairs of male connectors could be joined by connectors having pairs of recesses; but this is less preferred. Both direct linkage, and linkage via small linking pieces, are included in the concept of “connectors”. Direct linkage is preferred for ease of assembly.
Varying the diameter of interlocking multi-lumen tubular element 100 proximal to distal, and/or inserting diametrical transition type interlocking multi-lumen tubular element 100 features in conjunction with unique interlocking multi-lumen tubular element 100 designs to modify the relationship of the clear unobstructed central volume 36 to the peripheral instrument channel 119 volume at any point along the instrument.

The central tube with peripheral instrument channels (119) can taper up or taper down diametrically singly or in any combination or sequence along the instrument axial length, there can be transitional change in geometry from tubular to some other defined closed geometry perimeter even to the point of approximating a multifaceted polygon, square, rectangle triangle elliptical or any combination type of closed perimeter free form shape.

There can be peripheral lumen features that transition an instrument off axis, to guide or aim the instruments residing within, at a general axial deflection angle from the central axis at the instrument distal end Such off an axis delivery may be achieved in the design and position of the lumen feature element where the instrument is required to exit and/or may also be achieved by using a combination of a standard lumen element and a more distal element with a deflecting type of surface which is in alignment with the peripheral lumen itself. Such constructs thus could provide a means for peripheral instruments to exit the lumens at any point along the instrument axial length. The ideal embodiment for instruments residing within the peripheral lumens of this preferred embodiment is defined as embodiments generally with a length to diameter ratio of greater than 100-1 and a diameter of 3 mm or less. Such embodiments are most easily suited for an off axis deployment in this fashion for the function of the instrument described herein. However, many different embodiments and sizes can be axially deflected successfully provided that the net bend radius at the point of deflection is sufficiently large enough such that the instruments material remains in the elastic state through said bending area and does not cause a permanent deformation as a result of passage through the bending geometry and the overall friction of passage through the bending geometry is reasonable with respect to generating an axial force for movement.

A critical design constraint requirement for device function is that the assembled constructs that form the multifunctional instrument introducer deliver the instruments residing within the central and peripheral lumens with free axial sliding capability along the intended design path while allowing for the multifunctional embodiment to flex and bend in a controlled way without generating interference or preventing the control and positioning of said instrument that reside within. The ability to essentially snap fit the outer lumen constructs into the open peripheral instrument channels (119) and then close said channels with the outer sheath (190) after the instruments have been put in place, makes the assembly very easy regardless of the path of the instruments within the device. Controlling the shape and interface of the peripheral element lumens at the bending junction (104) will by design provide the needed clearance to alleviate any binding of instruments residing within during flexure.

The design of interlocking multi-lumen tubular element 100 allows a series of interlocking multi-lumen tubular element 100 components to be easily assembled one to the next in a daisy chain like manner. Such designs and assembly methods are preferred embodiments, and provide a significant advantage in setting the device configuration, the cost and manufacturing ease of the device. These components may be fabricated by numerous processes using materials well known in the art, such as but not limited to injection molding, cast molding, or extrusion for creating polymeric constructs, and metal injection molding or metal casting for generating metallic constructs.

In a preferred embodiment of the present invention, the materials used to generate interlocking multi-lumen tubular elements 100 are preferably made from the engineering thermoplastic materials class with properties of modulus and elasticity similar to but not exclusively from the nylon family of thermoplastics.

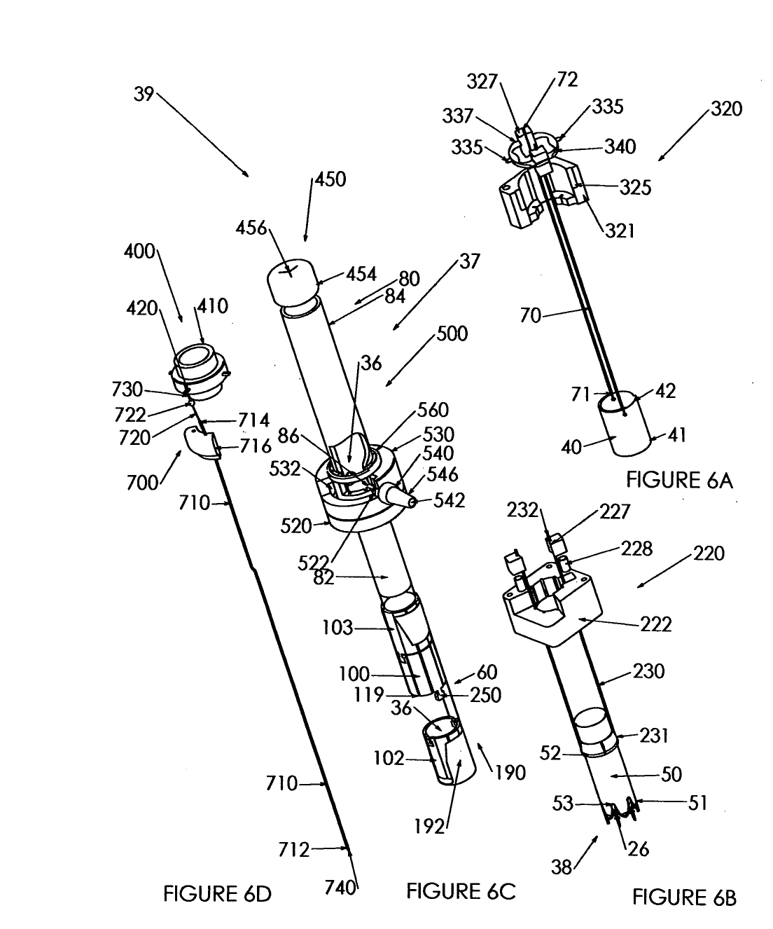

FIGS. 6A, 6B, 6C and 6D a series of exploded views illustrates additional features and relationships of the functional sub assemblies shown in FIG. 3 and has cut away sections of a number of elements to reveal and define internal features. Multifunctional instrument introducer 39 is shown in the shorter length embodiment of FIG. 3 which maintains all functional aspects of the preferred embodiments shown in FIGS. 1 and 2 and is comprised of a multifunctional instrument introducer distal end detail 38, and a multifunctional instrument introducer control end 37. Endoscope delivery tube assembly 60 in the central portion shown in this view is comprised of just two interlocking multi-lumen tubular elements (100) one of which is hidden to further reveal the position and function if instruments and control features which include: pull wire 70, length control wire 230 and a single embodiment illustration of a quadrant configured array of suture “t” stay needles 710. These embodiments are located within the peripheral instrument channels 119 of interlocking multi-lumen tubular element 100, the features of which have been illustrated in FIG. 4A, FIG. 5C and FIG. 5D respectively.

An interlocking multi-lumen tubular distal transition element 102 resides at endoscope delivery tube assembly distal end 61 and an interlocking multi-lumen tubular proximal transition element 103 resides at endoscope delivery tube assembly proximal end 62. Following the general description sequence used in FIGS. 1, 2, and 3, moving distal 38 to proximal 37 on multifunctional instrument introducer 39, shell element 40 is sealably sliding on tubular connecting element 50 which is sealably engaged with interlocking multi-lumen tubular distal transition element 102 at endoscope delivery tube assembly distal end 61.

Referring to FIGS. 6A, 6B 6C and 6D showing the functional sub assemblies of the instrument in exploded view multifunctional instrument introducer distal end detail 38, shell element 40 is reveal a self closing tissue fastener 26, an embodiment with functional aspects and a deployment scheme as described in U.S. patent application Ser. No. 11/728,569, LaBombard, filed Mar. 26, 2007, which is incorporated herein in its entirety by reference.

The self closing tissue fastener 26 is residing within shell element 40 at its distal end 41, with self closing tissue fastener 26 nested and engaged with the self closing tissue fastener profile feature 53 located on the tubular connecting element distal end 51 of tubular connecting element 50. Shell element proximal end 41 is connected to pull wire distal end 71 of pull wire 70 which resides within a peripheral instrument channel 119 of interlocking multi-lumen tubular elements 100 and runs the distal to proximal length of endoscope delivery tube assembly 60, terminating at self closing tissue fastener firing collar assembly 320 residing slideably on control end tubular member 80. Tubular member 80 is also shown in cutaway view to reveal the clear unobstructed central volume 36 which runs from the introducer control end 37 to the introducer distal end 38. In the preferred embodiment there are two identical pull wires 70 placed in a symmetrical axial orientation about the instrument axis at about 180 degrees apart. Such an orientation construct provides a distinct advantage in device performance which will become clear as the embodiment and control scheme is described in more detail below.

Self closing tissue fastener firing collar sub assembly 320 (FIG. 6A) is comprised of firing collar 321, pull wire compensation plate 340, and symmetrically located pull wire sliding locks 327 which align and orient with the pull wire 70 locations as previously described. As described previously in FIGS. 5A, 5B, 5C and 5D the endoscope delivery tube assembly 60 with multiple interlocking multi-lumen tubular elements 100 has the ability to flex along multiple pivot axes to generate a needed curvature. Such flexure as previously described above will by design vary the interlocking multi-lumen tubular element pivot gap 104.

One skilled in the art can appreciate that as this flexure occurs at each joint of the interlocking multi-lumen tubular elements 100, the actual lengths of each instrument channel 119 within endoscope delivery tube assembly 60, as related to a measurement from a fixed location on control end tubular member 80, to a fixed location on tubular connecting element 50, can vary depending upon the amount of total curvature of the multifunctional instrument introducer 39.

Conversely, the length as measured along the axial centerline of the clear unobstructed central volume 36 as taken from the exact same location on control end tubular member 80, measured to the exact same location on the tubular connecting element 50 previously defined is by design a constant length regardless of the instrument curvature.

Furthermore, in describing the relative differential length of a pair of instrument channels 119 which are by design located symmetrically positioned about the axial centerline of the clear unobstructed central volume 36, the relative length difference of each instrument channel 119 length is therefore equal and opposite. The amount of this difference is a resultant of the total amount of curvature of the instrument in a plane that is defined by the instrument channels 119 and the axial centerline of the clear unobstructed central volume 36, which all reside by design in a single plane running the axial length of the instrument. Said plane for the purposes of this submission is defined and described as the “neutral bending plane”.

Thus, instrument curvature in the neutral bending plane will result in an equal and opposite difference in lumen length as compared to the axial centerline length. Instrument curvature at 90 degrees to the neutral bending plane will result in no difference in the lumen length as compared to the axial centerline length.

A simple illustration to assist the reader in understanding the concept of off axis peripheral lumen length difference and the need for compensating for this effect when tubular type designs are bent into a curved state, is to take a simple straight length tube of flexible material with two opposing peripheral channels residing in the wall of the tube and bend it into an arc or circle placing the tube on a table top and keeping the peripheral channels parallel to the table top.

The tubular material for this illustration is by design flexible enough that the length of the centerline axis is constant and not changed as it transitions from the straight state to the bent state. The table top the bent tube is resting on represents the neutral bending plane in the previous discussion. The instrument lumens as described in the preferred embodiment of element 100 now reside “in the wall” of the tube in a location parallel to the table top.

In the bent state, the measured the arc length along the outer circumference of the tube (in effect the peripheral lumen length following along the outer arc), is now longer in pathway than the measured arc length along the inner circumference of the tube (the peripheral lumen following along the inner arc).

In the function of the preferred embodiment, this condition is achieved at each element 100 interface as the interlocking multi-lumen tubular element pivot gap 104 increases along the outer circumference and decreases along the inner circumference respectively.

Retuning the tube to an axial straight condition, each peripheral lumen is now the same length and equal to the central lumen axial length. Bending the tube in an arc in the opposite direction thus reverses the relative lengths of the lumens respectively.

One skilled in the art can now appreciate the effect of tube bending on peripheral lumen length. Placing rigid connections attached to each end of the simple tube example such as elements 40 and 80 in the preferred embodiment and attaching a pair of fixed length wires such as element 70 residing within the peripheral instrument channel 119 of the simple tube example to said rigid connection (40) residing at one end, and then projecting said wires (70) a fixed distance from the second end of the simple tube example in the axially straight condition establishes a fixed equal distance of both wires (70) from the second end.

Then, when the tube is now bent as described on the table top, the relative lengths of the wires (70) will now change as the circumference arc length of the inner bend curve and outer bend curve diverge equally and opposite from the fixed known axial length measurement. Therefore the lengths of the projecting wires (70) in relation to the simple tube example second end are also changing with respect to each other as a function of bending.

To control accurately any distal positioned element from a proximal control point such as assembly 320 described in this application, there is a need to compensate for this ever changing and varying length of off axis placed control features as a result of device bending. Such a compensating mechanism for control of distal features will now be further described.

To control the position and deployment of instruments within the peripheral instrument channels 119, therefore, it is highly desirable to be able to position, lock and actuate these instruments using embodiments located at or on the control end tubular member 80, regardless of the general path or curvature of the overall instrument and the effect such curvature has on the operating length of said instruments located within the peripheral instrument channels (119).

Such a length compensation scheme has been devised to overcome the varying length peripheral instrument channel attribute. This compensation mechanism is described as follows, with reference to FIG. 6A:

The function of the pull wire pivot plate 340 and interfacing geometries residing within firing collar 321 is to provide for a means of positioning, securing and actuating shell element 40 regardless of the overall profile and curvature of the of the multifunctional instrument introducer 39. Any motion generating instrument curvature changes the relative position of one pull wire proximal end 72 in relation to the other located symmetrically on the instrument. This available compensational ability allows the surgeon to lock the axial location position of firing collar 321 at the instrument proximal end, which in turn locks the axial position of shell element 40.

In detail:
Pull wire sliding lock 327 is engaged and secured to pull wires 70 at the pull wire proximal end 72.
Pull wire sliding lock 327 is slideably mounted on control end tubular member 80 and engages pull wire compensation plate 340, with connections such that the pull wire sliding lock 327 is able to freely move with an axial motion along the center axis of control end tubular member 80 while remaining engaged to the pull wire compensation plate sliding lock drive feature 337.
Pull wire compensation plate 340 can rotationally pivot about pull wire compensation plate pivot 335 which rotationally engaged and axially constrained to firing collar pivot 325.

That is, the preferred embodiment of the present invention provides a means for setting and maintaining a fixed axial location for peripheral instruments with regard to the multifunctional instrument introducer distal end detail 38 and more specifically, in the preferred embodiment, the physical location of shell element distal end 41 as related to the tubular connecting element distal end 51 location is controlled for the purpose of securing and firing self closing tissue fastener 26.

This position relation can be maintained and controlled regardless of device curvature or flexure during use. Furthermore, this positional relationship and control mechanism may be utilized for manipulating any instruments, singly or jointly, which may reside within the peripheral instrument channels 119.

In FIG. 6B, detailing the distal collar assembly 220, the proximal end 52 of tubular connecting element 50, which is sealably engaged to endoscope delivery tube assembly distal end 61, is attached to a pair of symmetrically oriented length control wires 230 at their distal ends 231. The wires 230 reside within peripheral instrument channels 119 of interlocking multi-lumen tubular elements 100 and run the length of endoscope delivery tube assembly 60, terminating at the distal collar assembly 220. Distal collar assembly 220 is sealably attached to endoscope delivery tube assembly proximal end 62 and control end tubular member 80, and is comprised of distal collar 222 shown in a cutaway view, a pair of length control sliding locks 227, and length control sliding lock spring 228 embodiments.

Length control sliding lock 227 is attached to the length control wire proximal end 232.

The location of length control wire 230 and the position of length control sliding lock 227 engages a length control sliding lock spring 228, such that flexure or curvature of the instrument (which, as previously detailed, generates a differential axial length relationship for mirrored symmetrical features residing within peripheral instrument channels 119), can maintain a spring tension force on endoscope delivery tube assembly 60 regardless of instrument curvature or path.

An alternative embodiment, not shown, which also enables securing the distal and proximal ends of endoscope delivery tube assembly 60 during flexure, includes pivoting features and wire engaging slides similar to the general configuration construct described above, that was used in this embodiment for position and control of the self closing tissue fastener firing collar assembly 320, which enables manipulation of shell element 40. Such an embodiment would include modifications and added elements, like those shown in assembly 320, for the purpose of generating an axial spring like tension force on pull wire compensation plate 340 by applying the tension force member at the interface of pull wire compensation plate pivot 335 and firing collar pivot 325 respectively.

A further enhancement to this embodiment would include user manipulated control features attached to pull wire compensation plate 340 to selectively tension or move length control wires, such as wires 230, thus providing a directional bending or steering function to the instrument. The axial motion of the pull wire changes the interlocking multi-lumen tubular element pivot gap 104 shown in FIG. 5A for the interlocking multi-lumen tubular element 100, thereby inducing bending.

Such a manipulation scheme would be best for interlocking features which are located about 70 to 90 degrees (in rotation) from the peripheral instrument channels 119 where the length control wire 230 elements reside. In FIG. 6B the optimal location in this embodiment is defined as the orientation of interlocking location 250 shown on the interlocking multi-lumen tubular element 100 as related to the approximately 90 degree position of length control wires 230 residing the in peripheral instrument channels 119.

One skilled in the art may now easily conceptualize any number of constructs, arrangement of features, selection of material compositions either singly or multiple coupled with control schemes which include the compensation geometry and control mechanism typical of that described herein. Such embodiments could be used in the design of a multifunctional instrument introducer with multiple user activated directional control features to provide for specific device attributes and performance.

In FIG. 6C, a rotary vacuum assembly 500 similar in design and function to known art used for vacuum or pressure energy transfer with unlimited rotational motion such for example that illustrated in U.S. Pat. No. 6,186,509 FIG. 4 and FIG. 5, is shown. It is comprised of a rotary vacuum mount collar 520 mounted sealably on control end tubular member 80 and includes peripheral instrument channels 119 which match in identical location and rotational orientation to the peripheral instrument channel 119 features of the endoscope delivery tube assembly 60.

Rotary vacuum mount collar 520 includes a pair of rotary vacuum mount collar ports 522 which are aligned with the control end tubular member vacuum port 86 features on the control end tubular member 80, thus providing an access pathway to the clear unobstructed central volume 36 for vacuum energy to be applied.

Mounted sealably and axially on rotary vacuum mount collar 520, is a rotary vacuum rotation collar 530 with a rotary vacuum hose connector 540 and a rotary vacuum clamp collar 520. Rotary vacuum rotation collar 530 is able to freely move a full 360 degrees in a sealed condition unimpeded while engaged sealably with rotary vacuum mount collar 520 and rotary vacuum clamp collar 520 respectively.

Rotary vacuum rotation collar 530 includes a defined annular rotary vacuum rotation collar vacuum space 532 which regardless of rotational position, allows a clear internal pathway from the rotary vacuum hose barb port 542, rotary vacuum rotation collar vacuum space 532, then through the rotary vacuum mount collar ports 522, with matched tubular member vacuum port 86 features to the clear unobstructed central volume 36 of the instrument for the purpose of providing vacuum energy. A rotary vacuum hose barb 546 connection feature is defined on the rotary vacuum hose connector 540 for connecting a vacuum energy delivery hose.

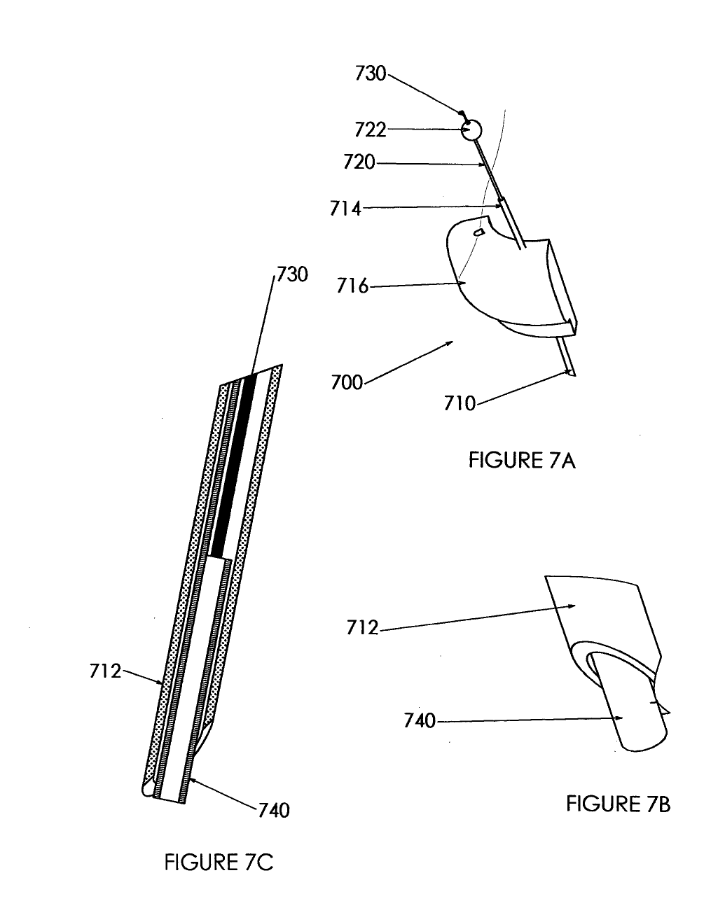

Referring to FIG. 6D, and FIGS. 7A and 7B which are enlarged details of the proximal and distal features of a suture “t” stay needle assembly 700:

Suture “t” stay needle assembly 700 is comprised of a hollow suture “t” stay needle 710 with a suture “t” stay needle proximal end 714 located generally at the control end tubular member proximal end 84, and a suture “t” stay needle distal end 712 located generally at the multifunctional instrument introducer distal end detail 38 location.

Residing within hollow suture “t” stay needle 710 at its distal end 712 is a suture “t” stay 740, with a suture “t” stay suture string 730 attached which runs the length of suture “t” stay needle 710. Inside the needle 710 is a “t” stay push wire 720, the distal end of which is in contact with the suture “t” stay 740 residing within. Push wire 720 has a proximal end which is terminated by a push wire control 722 feature, located at the suture “t” stay needle proximal end 714.

Suture “t” stay suture string 730 extends beyond the suture “t” stay push wire control 722, and can be secured and tensioned by the proximal suture collar suture anchor 420 which is located on the proximal suture collar 410 of the proximal suture collar assembly 400 securely and sealably positioned at the control end tubular member proximal end 84.

FIG. 6D shows a single suture “t” stay needle assembly 700, one of four in a preferred embodiment, in the fully retracted position. The suture ‘T’ stay needle assembly 700 can be manually manipulated to create an axial motion distal and proximal which will extend and retract the suture “t” stay needle 710 tip in relation to the position of the shell 40 at the most distal point of the instrument.

Referring to FIG. 7A, 7B and cross sectional view 7C for more detail, suture “t” stay needle deployment slide 716 is attached to the suture “t” stay needle 710 at suture “t” stay needle proximal end 714. Suture “t” stay needle deployment slide 716 is moveable in an axial direction along the outer surface of control end tubular member 80. Such movement controls the deploy and retract action of the suture “t” stay needle 710 and all associated components residing within a peripheral instrument channel 119.

The length of suture “t” stay needle 710 is designed to place the suture “t” stay needle distal end 712 slightly proximal to the tubular connecting element proximal end 52 when the “t” stay needle deployment slide 716 is located in its most proximal location. In this position, suture “t” stay needle 710 resides within the peripheral instrument channel 119 of distal transition element 102 and is hidden by outer sheath 190 which covers the suture “t” stay needle 710 in a sheath like manner and prevents the suture “t” stay needle 710 from engaging tissue inadvertently or causing tissue damage during the placement or movement of the instrument in the surgical field. (Deployment of the T-stay will be described below.)

In FIG. 7B, endoscope seal 450 is comprised of a endoscope seal distal end 454 engaged sealably and securely with control end tubular member proximal end 84 of control end tubular member 80 and an endoscope seal instrument access feature 456 located axially central on the proximal end of endoscope seal 450.

Endoscope seal instrument access feature 456 is designed both geometrically and by material specification to allow endoscopic instruments of numerous sizes to pass through the embodiment and into the clear unobstructed central volume 36 of the instrument while still maintaining a seal adequate for generating a vacuum force within the central space. Materials and geometric designs which are useful for creating this embodiment feature and function are well known in the art and may consists of radial slits, annular corrugations or similar features, elasticity and lubricity of seal 450, or a combination thereof.

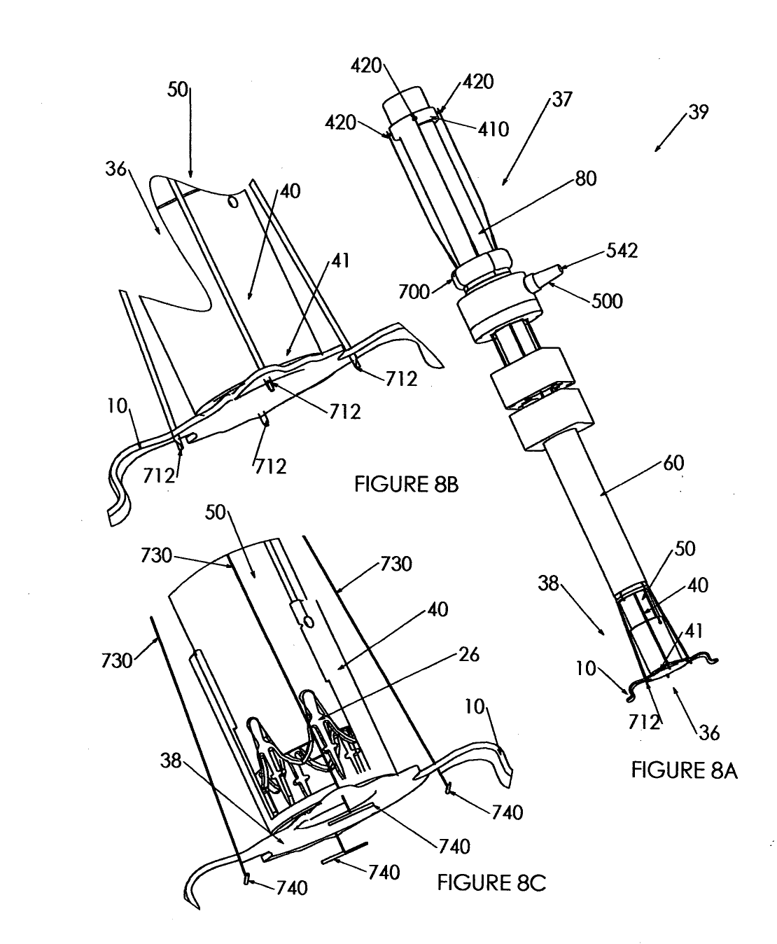

FIGS. 8A, 8B, 8C, FIGS. 9A and 9B and FIG. 10 will now be used to illustrate the use of the multifunctional instrument introducer to secure and maintain a clear unobstructed working channel to a target tissue site, for example as would be employed in the performance of a NOTES procedure. Also described is a means of effectively closing the incision made in the target tissue, upon the completion of the procedure.

FIG. 8A illustrates the multifunctional instrument introducer which has been moved into position to the target site, which is typically (but not limited to) a location within the gastroesophageal system, such as the stomach, where an incision is needed to pass an endoscope through and into the body cavity beyond to perform a NOTES procedure.

The target site represented by Tissue 10 has been located and is shown in contact with shell element 40. A self closing tissue fastener (not shown) is residing within shell element 40 at its distal end 41.

Vacuum energy is applied to the central volume 36 of the multifunctional instrument introducer 39, through the rotary vacuum hose barb port 542 located on rotary vacuum assembly 500, which freely communicates with the central volume 36 within control end tubular member 80 and endoscope delivery tube assembly 60. This vacuum energy secures the tissue 10 against the distal end 41 of the instrument allowing the peripheral instruments to interact with tissue 10 in a predictable manner.

Once the tissue is engaged and held securely, the suture ‘T’ stay needle assemblies 700 can be deployed into tissue 10. First, each suture “t” stay needle deployment slide 716 is axially displaced distally along tubular member 80. Referring to FIG. 8B, showing an enlarged view of the introducer distal end detail 38, the suture “t” stay 740 located at and within the suture “t” stay needle distal end 712 for each of the suture “t” stay needle assemblies 700 has penetrated into tissue 10.

Next, sliding the suture “t” stay push wire control 722, connected to the suture “t” stay push wire 720, in an axial motion toward the suture “t” stay needle proximal end 714 will eject the suture “t” stay 740 from the inside of the suture “t” stay needle distal end 712. This additional motion causes suture “t” stay 740 to penetrate into the tissue fully and allows the complete engagement of suture “t” stay 740 with tissue 10.

As illustrated in FIG. 8C, upon completion of the engagement of the suture “t” stay 740 with the tissue 10, the suture “t” stay push wire control 722 and suture “t” stay push wire 720 is then withdrawn from the instrument in the proximal direction, leaving the suture “t” stay 740 engaged in tissue 10 and the connected suture “t” stay suture string 730 axially deployed within the suture “t” stay needle 710, and able to manipulate, tension and secure tissue by controlling the tension and position of the suture “t” stay suture string 730 at the proximal end of the instrument. The vacuum can now be disengaged, and the clear unobstructed central volume 36 provides a sealed sterile pathway to the target site which can be securely maintained in its intended position on the tissue 10. Proximal suture collar suture anchors 420 located on proximal suture collar 410 are designed for suture “t” stay suture string and string tension management to maintain and secure the instrument at the target tissue site. These are conventional designs, and as such may have in their function any number of designs well known in the art that would adequately secure and tension sutures.

Next, after using the multifunctional instrument introducer of the present invention for providing a secure controlled access pathway to target tissue, a surgeon following the general outline of a NOTES procedure would pass instruments through the clear unobstructed central volume 36 to perform various operative procedures, including, without limitation, to incise the target tissue and open a passage through it; to pass instruments, endoscopes and the like through and into the body cavity to conduct a surgical procedure; and to monitor said procedure. Upon completion of the NOTES procedure, the surgeon may use additional functional embodiments of the present invention to close and secure the target tissue, to promote healing of said incision in the target tissue.

Once the tip of the device has been attached to the target tissue surface, various other operations and materials can be applied to the tissue surface via the introducer device. Either the peripheral lumens 119 or the central lumen 36 can carry devices for irrigation, drug delivery, cleansing and sterilizing liquids, fiber optics, electrocautery leads, heated cautery tips, grasping devices, cutting devices, and in general any of the many functional devices known in the art that can be passed through the approximately 0.5 to 3 mm diameter of a peripheral lumen 119, or the larger diameter of the central lumen 36.

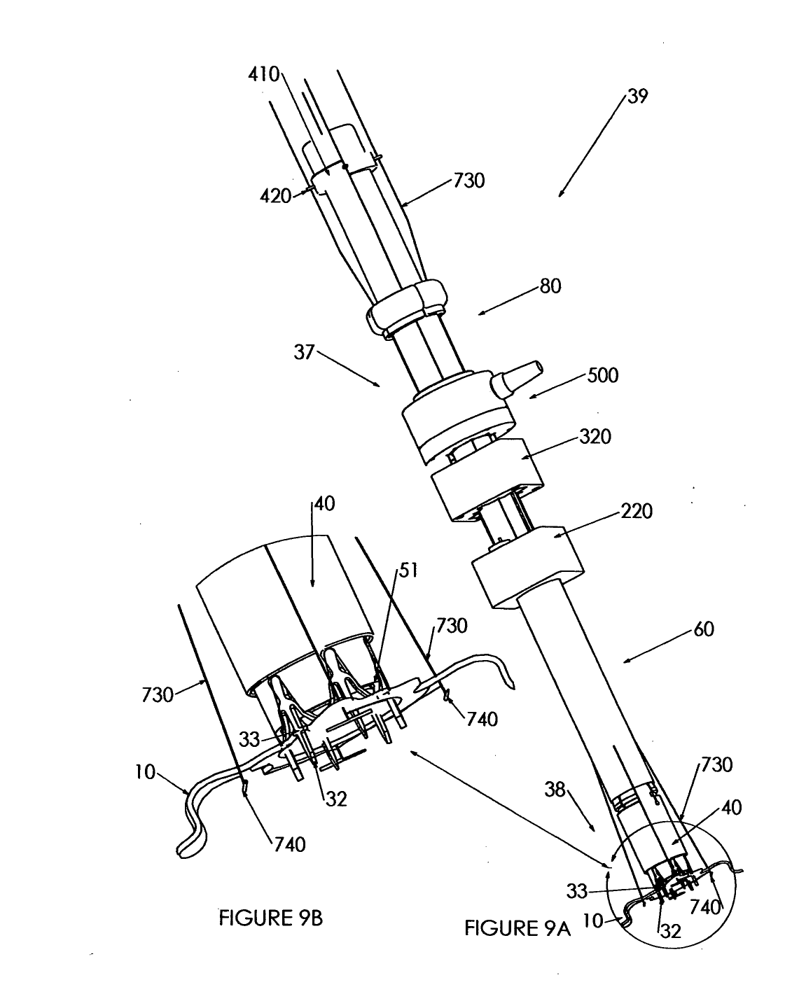

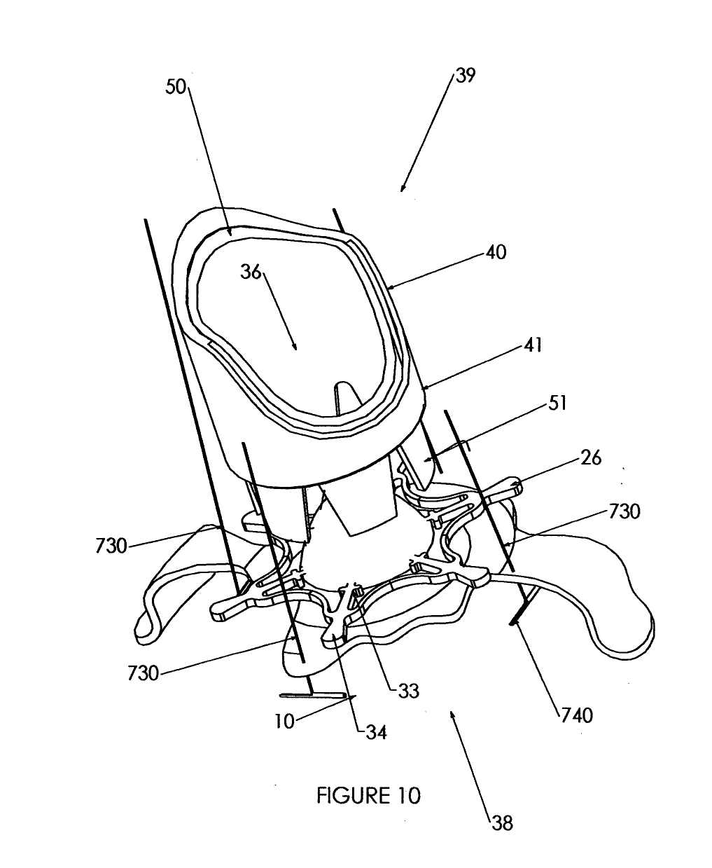

FIGS. 9A and 9B and FIG. 10 illustrate the procedure for performing such a closure using a self closing tissue fastener and tissue closing technique as described in U.S. patent application Ser. No. 11/728,569 “Self Closing Tissue Fastener”. Referring to FIG. 9A and FIG. 9B an enlarged detail of the distal instrument end, suture “t” stay 740 is now deployed into tissue 10 as previously described in FIGS. 8A, 8B and 8C. Suture 730 attached to “t” stay 740, and running the full length of the instrument, provides a means for the surgeon to tension tissue 10 to the multifunctional instrument introducer distal end detail 38. Vacuum is then applied through the rotary vacuum assembly 500 via the central volume 36, drawing the tissue up and into the clear unobstructed central volume 36, as the self closing tissue fastener firing collar assembly 320 and multiple suture “t” stay suture strings 730 are moved together in a distal to proximal direction?? proximally. This axial movement of shell element distal end 41, including suture “t” stays 740 and tissue 10, and completion of the axial movement of the self closing tissue fastener firing collar assembly 320's proximal stroke length, removes the constraining shell 40 from the tensioned tissue fastener 26, thereby deploying the self closing tissue fastener 26, which returns to a relaxed planar condition and thereby imbeds the fastener tissue piercing features 32 and fastener tissue stopping features 33 into tissue 10, thereby closing the opening.

Referring to FIG. 10, which is a truncated illustration of the distal end of the instrument, the self closing tissue fastener 26 is now fully deployed. The clear unobstructed central volume 36 has been maintained and unobstructed throughout the procedure so that endoscopes and the like can provide continual direct visualization to the surgeon of the site as the self closing tissue fastener 26 is actuated to close the incision. The array of suture “t” stays 740 with attached suture “t” stay suture string 730 are also still engaged with tissue 10.

As the multifunctional instrument introducer 39 is withdrawn from the surgical site, the array of suture “t” stay suture strings 730 remain, the proximal end of each suture string at a location outside the patient which was generally located at about the proximal end location of the instrument and is readily accessable for manipulation. Using techniques well known in the art, the surgeon can use remote suture securing apparatuses, fastener clips, and the like to secure the individual suture “t” stay suture strings 730 in a scheme to further secure the target tissue. Such a scheme if executed for example in an opposite corner pattern will pass directly across the tissue engaged central self closing tissue fastener 26. Such a pattern is advantageous in that it creates a primary and a secondary means of ensuring effective tissue closure thus providing a redundant highly secure closing mechanism.

Furthermore, such suture securing schemes may also include the use of medicated, medicament delivery or biomaterial wound healing aids which would be deployed and secured by the suture securing technique, further providing enhanced healing benefits to the patient.

### Materials for Construction

The designs of the embodiments of the present invention provide numerous opportunities to select materials and fabrication processes which are extremely cost effective while still providing the performance properties needed. Interlocking multi-lumen tubular elements (100) which by design can snap fit together, may be comprised of polymeric materials, composites or laminates which are light weight and durable, or conversely could be die cast metallic based ultra thin wall constructs with a-traumatic soft outer coatings and slippery lumen coatings or combinations thereof. Such constructs can be easily injection molded, metal injection molded or cast molded since the design of the multi lumen embodiment features and their relationship to the tubular geometry and central volume provides for a robust tool design and long tool life.

Control end tubular member (80), tubular connecting element (50), shell element (40), the distal collar assembly (220) components, the self closing tissue fastener firing collar assembly (320) components, the rotary vacuum assembly (500) components, the suture “t” stay needle deployment slide (716), and the proximal suture collar assembly (400) components currently in the preferred embodiment comprised of metals such as aluminum and stainless steel, may also be comprised of well known engineering thermoplastic materials which can use injection molding processes and tooling to generate consistent, robust, structural components which by design can have assembly engaging features, position locators, snap fitting embodiments and the like integral to the embodiment for further cost effective assembly.

In generating these components and assemblies, biological, drug, therapeutic and/or antibacterial coatings may also be employed on selected surfaces to aid and assist in maintaining a sterile field within the clear unobstructed central volume 36 of the instrument. Other such lubricious coatings may be employed for use within the peripheral instrument channels. In generating a sterile field, sterilizing substances may be introduced from the proximal end of the instrument after the distal tip of the instrument has been affixed to target tissue, to wash away or sterilize any contaminant.

Various embodiments and figures have been described in this specification to allow it to be understood by persons of ordinary skill in the appropriate arts. The scope of the invention is not limited to the specific embodiments described, but is limited only by the scope of the claims.

1. An introducer for introducing instruments to a site in the body, the introducer comprising:
a torqueable, bendable central tube made by joining together multiple cylindrical elements,
said elements being joined together by connectors at the ends of said elements to make a tubular assembly having a first, central lumen;
and having at least two second lumens in the outer walls of the elements, said second lumens open to the outside of the tube and aligned so that when the elements are assembled to form the tube 60, second lumens are formed which run the length of the segments, and optionally the length of terminating segments when present;
further comprising at least two wires pressed into a pair of said second lumens from the outside of the tube, said lumens being on opposite sides of the tube, and said lumens being located about 90 degrees from at least one pair of connectors;
and further comprising a constraint surrounding said central tube confining said wires in the peripheral lumens when the device is in use.

2. The introducer of claim 1, wherein the degree of bending that can be obtained per element is limited to a maximum angle per element of about 6 degrees or less.

3. The introducer of claim 1, wherein the tube can maintain internal sterility while being introduced into the body and bent.

4. The introducer of claim 2 wherein the angle is controlled by the relative lengths of the male and female connecting elements in the dimension running along the cylinder axis.

5. The introducer of claim 1, wherein there are more than two peripheral lumens, and at least two lumens are used to do one or more of controlling the release of a fastener, controlling the tip position of the introducer relative to tissue by the use of T-stays, introducing fluids to or through a tissue surface, and introducing devices to or through a tissue surface.

6. The introducer of claim 1, wherein said constraint means are selected from one or more of shrink wrapping; adhesive tape; polymeric and metallic mesh, braid, coils and bands, optionally including an airtight layer; and tubing cast in place.

7. The introducer of claim 1, where a tubular assembly has more than one symmetrical pairs of second peripheral lumens in the walls of the cylindrical elements.

8. The introducer of claim 1, further comprising an operative instrument within the structure of said introducer.

9. The introducer of claim 8, wherein the operative instrument is selected from one or more of:
one or more tissue-fastening devices which can be deployed from the distal region of the introducer to fasten tissue to itself; and
one or more stylets for placing tissue anchoring means into tissue adjacent the distal end of the instrument.

10. The introducer of claim 9, wherein the tissue anchoring means serve to provide at least one of a means to stabilize the position of the distal tip of the instrument with respect to the tissue, and to provide means for introducing a tissue fastener to the affected tissue after the introducer is withdrawn from the operative site.

11. The introducer of claim 10, wherein the tissue anchoring means are T-stays.

12. The introducer of claim 6 wherein the constraint means are sufficiently airtight to allow the use of vacuum inside the central lumen of the introducer.

13. The introducer of claim 2 wherein the constraint means are sufficiently sealed against air and bodily fluids to permit the central lumen of the introducer to remain in an initially sterile condition during at least one procedure.

14. The introducer of claim 1 wherein the introducer is constructed so that it can freely bend in at least one plane with wires running in peripheral lumens aligned with said plane.

15. The introducer of claim 1 wherein the introducer is constructed so that it can freely bend in more than one plane with wires running in peripheral lumens aligned with each of said more than one planes.

16. The introducer of claim 1 further comprising a pull wire compensation plate to automatically adjust the effective length of pull wires so as to correct for bending in the introducer.

17. The introducer of claim 1 wherein the introducer is torqueable, as defined herein, without requiring means for longitudinal compression.

18. The introducer of claim 8 wherein at least one operative instrument carried in the peripheral channels can be deflected away from the longitudinal axis of the introducer when the operative instrument is advanced distally.

19. The introducer of claim 1 having sufficient rigidity to provide precise control of the position of the distal end of said introducer relative to the proximal end of said introducer regardless of the amount of bend that may be present in the instrument.

20. The introducer of claim 16, further comprising means for maintaining a fixed axial length irrespective of bend radius in more than one plane.

21. The introducer of claim 2, wherein the limited degree of bending at each joint allows the tubular elements to be essentially incompressible so that the length of the instrument along its central axis is constant.

22. A method for making and using a portal at a selected site in a tissue to allow the performance of one or more surgical procedures, the method comprising:
providing an introducer according to claim 1;
directing the introducer to the selected site;
affixing the introducer to the site by stays or functional equivalents thereof; and
delivering an instrument to open a portal if required; to perform procedures; and to at least partially close a portal if created;
wherein the tissue is selected from the delimiting tissue layers of one of the gastrointestinal tract, the reproductive tract, the urinary tract, and the respiratory tract including the sinuses.

23. A method of making a bendable and highly torqueable tubular introducer, the method comprising
providing cylinders carrying connectors, said cylinders being rigid;
axially linking said cylinders together, directly or via added linking connectors;
providing peripheral channels, extending at least the length of said linked cylinders;
providing at least two control wires for bending the instrument, located in at least two of said peripheral channels;
providing constraint means to confine said control wires in said channels; and
providing means for length compensation when the introducer is bend.
